(19) United States
(12) Patent Application Publication
(10) Pub. No.: US 2019/0105479 A1
(43) Pub. Date: Apr. 11, 2019

(54) DETACHABLE DILATOR WITH EXTRACTION DEVICE AND METHOD OF USE

(71) Applicant: SaiNath Intellectual Properties, LLC, Largo, FL (US)

(72) Inventor: Iyunni Venkata Sesha Sayi Nath, Seminole, FL (US)

(21) Appl. No.: 16/062,821

(22) PCT Filed: Dec. 15, 2016
(86) PCT No.: PCT/US16/66892

§ 371 Date: Jun. 15, 2018

Related U.S. Application Data
(60) Provisional application No. 62/267,567, filed on Dec. 15, 2015.

(51) Int. Cl. A61M 29/00; A61B 17/02; A61B 17/50; A61J 15/00

(52) U.S. Cl. CPC A61M 29/00; A61B 17/0218; A61B 2017/0034; A61J 15/0019; A61B 17/50

(57) ABSTRACT

A detachable dilator with an extraction device has a detachable dilator tip with a hole and an extraction device having a bead too large for passing through the hole and a string extending from the bead through the dilator tip. The string has a retrieval portion for coupling with a tool extending through an endoscope. For example, the retrieval portion may be a loop attached to the bead by the string. For example, the loop may be formed by knotting the string.

### CROSS RELATED APPLICATIONS

This application is a 371 U.S. National Phase of International Application PCT/US2016/066892 filed Dec. 15, 2016 which claims priority to filing date of U.S. Provisional Appl. No. 62/267,567, entitled DETACHABLE DILATOR AND EXTRACTION DEVICE AND METHOD OF USE, which was filed on Dec. 15, 2015, the specification and drawings of which are incorporated herein by reference in their entirety.

### FIELD OF THE INVENTION

The field relates to percutaneous endoscopic gastrostomy catheters.

### BACKGROUND

U.S. Pat. No. 8,057,429 issued Nov. 15, 2011 shows a feeding tube that is insertable using a introducer needle passing through the central channel of a percutaneous endoscopic catheter. The disclosure of this patent is incorporated by reference in its entirety herein for the purpose of showing a procedure for using such a catheter. This patent has a loop for securing the catheter in the patient, but it does not show any loop or any other device for retrieving a detachable dilator. U.S. patent application Ser. No. 12/905,522 was filed Oct. 15, 2010 which claims the benefit of and priority to U.S. Pat. Appl. Ser. No. 61/031,442, filed Feb. 26, 2008, and U.S. Pat. Appl. Ser. No. 61/113,697, filed Nov. 12, 2008, and U.S. patent application Ser. No. 12/393,717, filed Feb. 26, 2009. This application is incorporated by reference in its entirety for the purpose of showing how a detachable dilator may be used for insertion of catheter into a cavity in the human body. Nothing in this reference dislcoses the use of an extraction device with a detachable dilator for retrieval of the detachable dilator.

### SUMMARY

A detachable dilator and extraction device for percutaneous endoscopic catheterization comprise a detachable dilator and an extraction device for extracting the dilator when it is detached from a catheter after the catheter is inserted into a body cavity, such as the stomach. For example, the extraction device comprises a bead and a string, the bead being fixed onto the string and arranged with a detachable dilator such that, when the string is pulled by an endoscopically introduced device, the detachable dilator is secured to the tool for retrieval by the endoscope.

The term “bead” means any globular or cylindrical body, of any shape with a hole through it, through which a string may be strung. An outer dimension of the bead is greater than a dimension of a hole passing through a detachable dilator, such that the bead is incapable of passing through the detachable dilator, when the string is passed through the hole through the detachable dilator. The bead may be made of any solid material, such as a glass, plastic, ceramic or metal or any combination of these. In one example, the bead is made of a plastic, such as a silicone or urethane material. For example, a silicone or urethane tube may be cut to a length incapable of passing through the hole in the detachable dilator.

The term “string” means any elongated wire, suture, line, cord, thread, strip, fiber or the like, without limitation on form, which has an outer dimension capable of passing through the hole in the bead. The string may be a flat strip of material or may have any other cross section, such as a circular cross section. The material of the string has, preferably, a high tensile strength such that pulling on the string to retrieve the bead and the detachable dilator does not break the string and a high degree of flexibility such that string is capable of bending during retrieval through viscera, lumen and/or orifices. The degree of flexibility necessarily depends on the application. For example, a slack suture is preferred for tying knots, if knotting is required. Alternatively, a bead may be crimped or adhered onto a string and no knot may be required, which may allow a stiffer string to be suitable for some applications. For example, a suture may be used for the string having an average diameter from about 0.044 mm to 0.45 mm. In one example, a synthetic suture is used, such as a monofilament polypropylene, a braided polyester or a braided silk. The knot-pull strength, in kilograms, of a suture string in this example may be from 0.09 kg to 5.23 kg, for example.

The terms “knot” and “knotting” mean any of the suitable ways of tying a string. Many types of knots are known. For example, a double overhand stopper knot may be used to secure one end of a string from passing through a hole. A double overhand stopper knot may be considered a “bead” in this context, because it is a globular body with a hole through it, through which the string is strung. However, a bead that is not a knot may be preferred, because the outer diameter of a knot can change depending on how tightly the knot is tied. Some knots may tighten as force is applied to the extraction device, and such a knot may slip through the hole in the detachable tip. Therefore, a bead other than a knot may be preferred. Alternatively, a FIG. 8 knot may be used to secure one end of a string from passing through a hole, although this knot may not be as secure as the double overhand stopper knot, it may be sufficiently secure to function for the purpose of extracting the detachable dilator and extraction device.

A method of extraction using the detachable dilator and extraction device for percutaneous endoscopic catheterization comprises: disposing the detachable dilator and extraction device on the end of a catheter, such that the extraction device extends from the detachable dilator, the extraction device having a portion that is sized to interferringly engage the detachable dilator such that the portion cannot pass through a hole through the detachable dilator through which the extraction device extends; providing a retrieval portion of the extraction device that extends through the hole in the detachable dilator such as a loop or other point of attachment or retrieval that is capable of being gripped by a tool, such as a forcep, hook or snare passed through an endoscope; securing the retrieval portion, such as by using the tool; and pulling the detachable dilator and extraction device, wherein the detachable dilator and extraction device are extracted from body cavity or lumen, for example.

In one example, a catheter for percutaneous endoscopic catheterization for use with an endoscopically introduced device, comprises a catheter tube, a detachable dilator and an extraction device. The catheter tube has a central channel extending through the tube from a proximal end of the tube to a distal end of the tube. The detachable dilator has a central channel extending through the dilator from a distal end of the dilator to a proximal end of the dilator, opposite of the distal end of the dilator, the proximal end of the dilator fitting onto the distal end of the tube such that the central channel of the dilator aligns with the central channel of the tube. The extraction device comprises a string and a bead. The string has a proximal portion and a distal portion. The bead is fixed on the proximal portion of the string. The proximal portion of the string and the bead may be arranged within the catheter at the proximal end of the dilator, opposite of the distal end of the dilator, and at least a portion of the distal portion of the string extends from the distal end of the dilator. For example, the bead retains the proximal portion of the string within the dilator when the at least a portion of the distal portion of the string is secured by an endoscopically introduced device, such as a hook, forceps or the like, for extraction of dilator. For example, the bead has a body and a hole extending through the body through which the proximal portion of the string extends. The body of the bead may be cylindrical, and the hole may extend through the body along a longitudinal axis of the cylindrical body. For example, the string may be strung through the hole in the body of the bead. An outer dimension of the bead may be greater than a dimension of the central channel extending through the dilator, preventing the bead from passing through the central channel of the dilator. In one example, the bead is made of silicone.

For example, the string may be an elongated wire, a suture, a line, a cord, a thread, a strip, or a fiber. The string may loop or may have a loop, knot or a second bead formed on the distal end of the string to help secure the string to an endoscopically introduced device. For example, the string may be made of a suture material. The string may have an average diameter from 0.044 mm to 0.45 mm. The suture material may be knotted such that a knot fixes the string on the bead. The knot-pull strength of the string may be from 0.09 kg to 5.23 kg.

In one example, the bead may be crimped or adhered onto the string, and the string may be not knotted. Alternatively, the string may be knotted, and the knot may fix the bead on string, or the bead may be fixed by a combination of the knot and an arrangement of the bead, string and dilator.

If a suture material is selected for the string, the suture material may be of a monofilament polypropylene, a braided polyester or a braided silk. In one example, the bead may be formed by knotting the string to secure the string through the dilator. In one example, a double overhand stopper knot may be selected to either form the bead or to secure bead on the string. Alternatively, the body of the bead is not formed by knotting the string.

A method for extraction of the dilator of the catheter may comprise endoscopically introducing the endoscopically introduced device, such that the endoscopically introduced device is disposed adjacent to the dilator; securing the distal portion of the string extending from the dilator to the device; and withdrawing the endoscopically introduced device, the dilator and the extraction device from the patient.

### BRIEF DESCRIPTION OF THE DRAWINGS

The following drawings are illustrative examples and do not further limit any claims that may eventually issue.

FIG. 1 illustrates a perspective view of an example of a bead extraction device.

FIG. 2 illustrates a perspective view of another example of a bead extraction device.

FIG. 3 illustrates a perspective view of an example of a bead extraction device disposed in a feeding catheter with a detachable dilator.

FIG. 4 illustrates a perspective view of another example of a bead extraction device with a detachable dilator, removed from the feeding catheter (outer diameter of the removed feeding catheter shown in dashed lines).

FIG. 5 illustrates yet another example of a bead extraction device with a detachable dilator.

FIG. 6 illustrates a side plan view of a detachable dilator.

FIG. 7 illustrates a perspective view of a detachable dilator.

FIG. 8A illustrates a back end plan view of the detachable dilator of FIGS. 6 and 7.

FIG. 8B illustrates a tip end plan view of the detachable dilator of FIGS. 6 and 7.

FIG. 9 illustrates an example of a tool for use in retrieving a bead extraction device and detachable dilator.

When the same reference characters are used, these labels refer to similar parts in the examples illustrated in the drawings.

### DETAILED DESCRIPTION

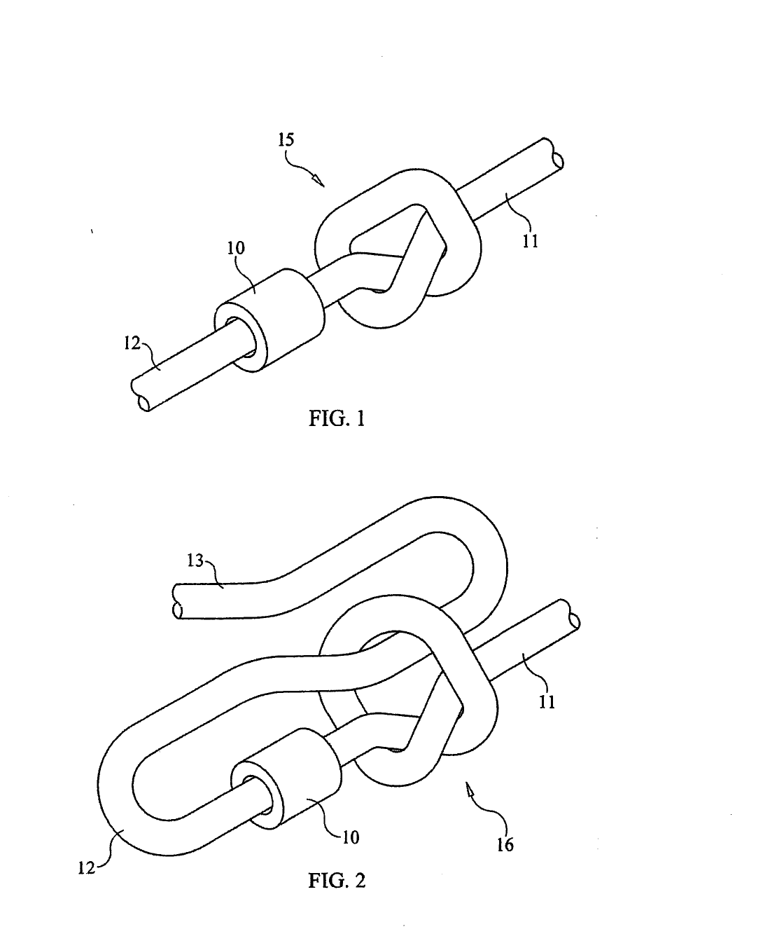

In one example, the extraction device comprises a 3 millimeter outer diameter spherical glass bead with a 0.1 millimeter through hole, passing through the spherical glass bead, and a 0.044 mm average diameter polypropylene monofilament suture having a length of 20 cm with its two free ends passing through the hole in the bead, forming a loop on one side of the hole through the bead. On the opposite side of the hole through the bead, the free ends are knotted, such that the knot formed cannot be pulled through the hole through the bead. The loop of the extraction device may be passed through a hole through the detachable dilator, such that the loop extends from the detachable dilator, when the detachable dilator is disposed on a catheter. In one example, a knot may be tied in the loop adjacent to the bead, such that the loop cannot pass back through the bead. In an alternative embodiment, a thicker string can be used, such as a 0.09 millimeter average diameter string, and each free end may be tied. One proximal free end may be passed through the hole in the bead and tied with a knot, and the opposite, distal end may be tied with a loop after passing the extraction device through the hole in the detachable dilator, for example. The free ends of the string 12 may be tied with a knot 15, such as the simple knot illustrated in FIG. 1, or may use a knot known for not pulling through a hole, such as a double overhand stopper knot or a FIG. 8 knot. There are too many knots to show all of the knots that could be used for this purpose here, and the knot 15 represents all of these types of knots, as a representative knot.

FIG. 2 illustrates a way of securing a bead on a string without using separate knots on each side of the bead 10. In this example, the bead 10 is a length of silicone or polyurethane tubing. A knot 16 is formed in a proximal end 11 of the string 12, which has been passed through the hole in the bead 10. By passing the opposite, distal end 13 of the string through the knot 16, a loop is formed by the string 12. This loop may be pulled until the bead 10 is secured in place, for example. The distal end 13 may be tied to the proximal end 11, such as by using any of a variety of knots 15.

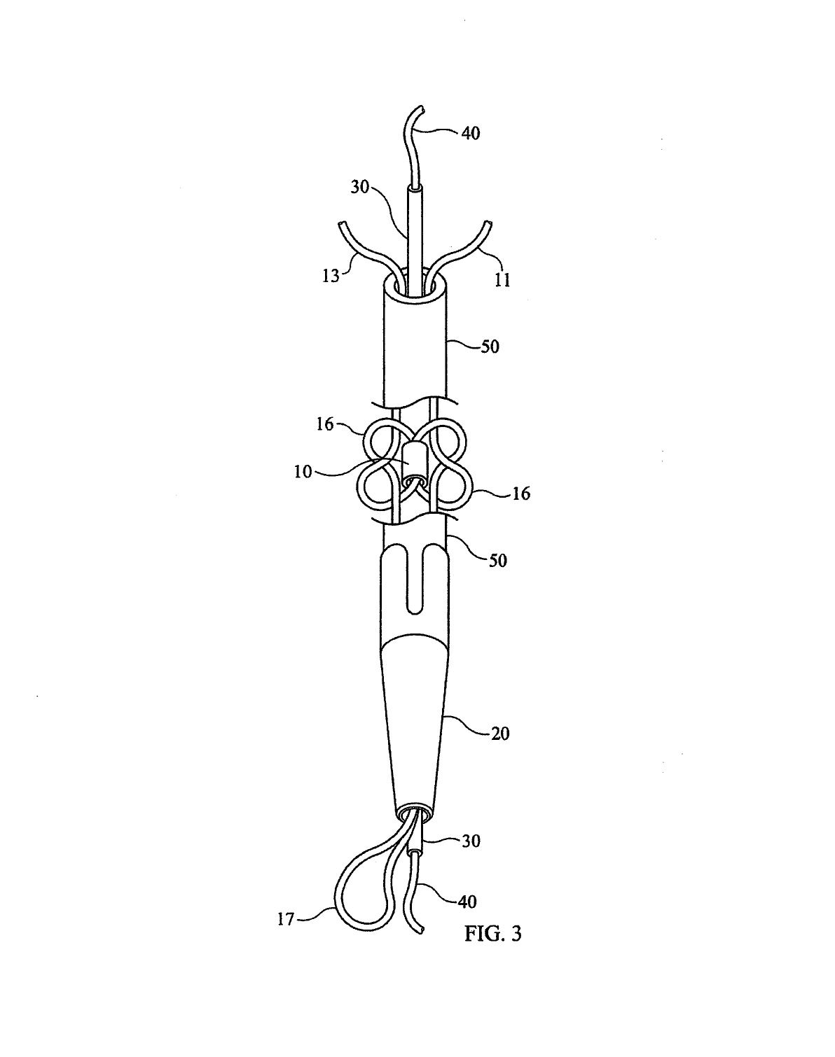

FIG. 3 illustrates an example where both free ends 11, 13 of the string 12 are disposed on the same side of the detachable dilator 20. The loop 17 is formed by a portion of the string 12 between the two free ends 11, 13. The knot 16, in this example, secures the bead 10 and is formed by how the two ends 11, 13 are knottingly engaged with each other and through the bead 10. Additional securing knots may be tied by the free ends 11, 13, such as half hitch knots and hitch knots. The loop 17 may extend through the hole 24 (FIG. 8A) through the detachable dilator 20, for example, providing a retrieval point that is easily engaged by a hook, snare or forceps passed through an endoscope. The loop 17 does not impair movement of the stainless steel introducer needle 30 illustrated in FIG. 3, because the hole 24 is large enough in diameter to accommodate both the needle 30 and the loop 17, for example.

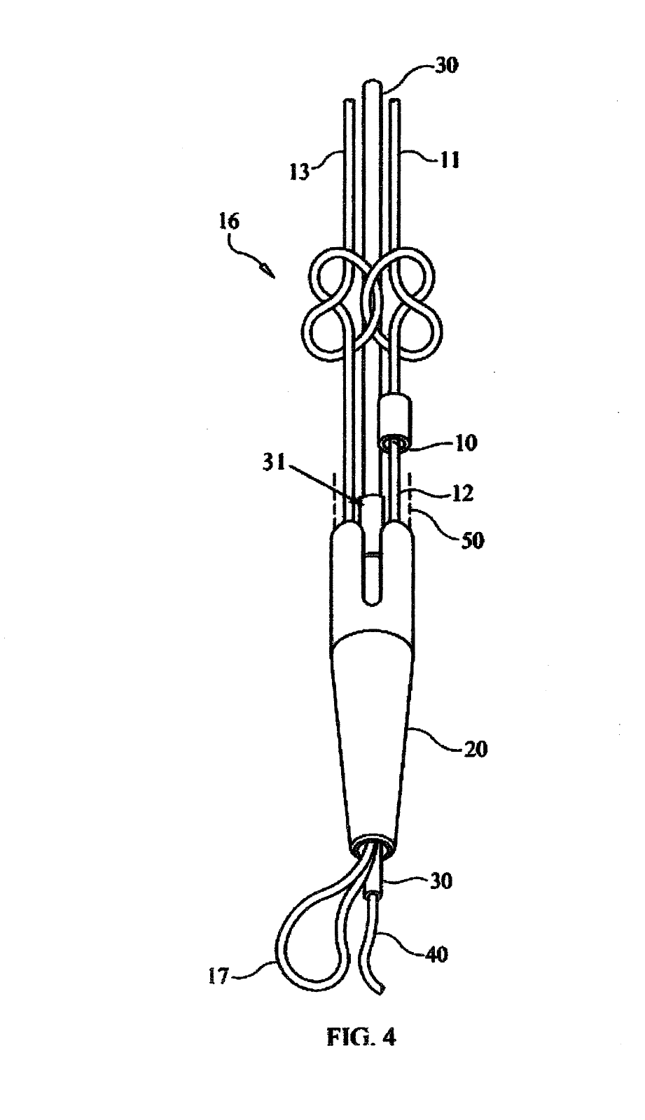

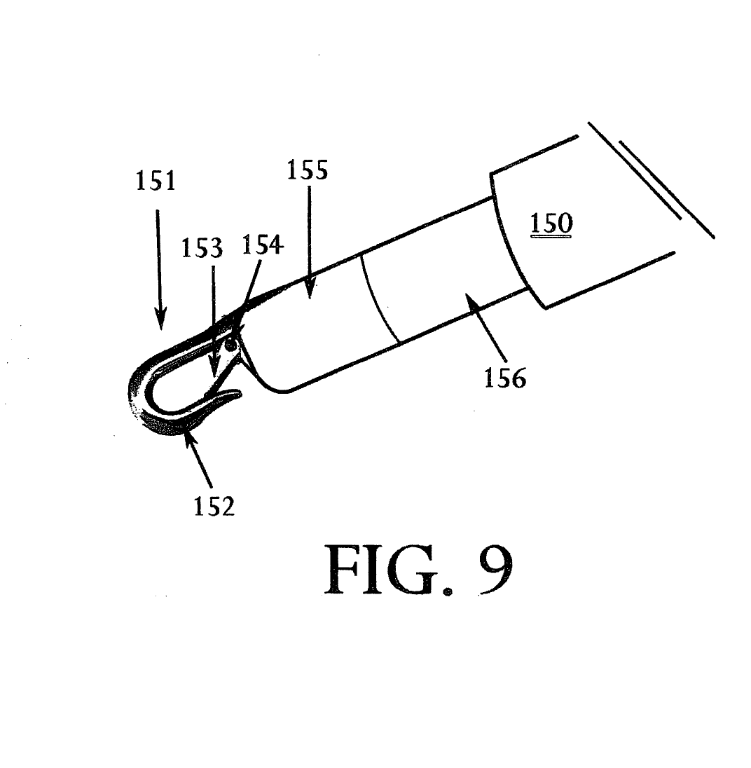

FIG. 4 illustrates another example. In this example, the bead 10 is not part of the knot 16, and the bead is not secured entirely by the knot 16. The knot 16 prevent the bead 10 from being removed from the loop 17 formed in the string 12. In this example, the loop 17 is passed through the hole 24 in the detachable dilator 20, and the bead 10 is sandwiched between the knot 16 and the detachable dilator 20. In this example, the dilator has been detached from the catheter 50. The needle 30 and guide wire 40 remain in place only to provide a frame of reference for the drawing. Ordinarily, the needle 30 would be withdrawn from the dilator 20 and the body of a patient before the catheter is withdrawn. However, in one example, the needle 30 is used to push the dilator 20 off of the catheter 50. For example, a portion of heat shrinkable tubing 31 may be secured on a portion of the outer periphery of the needle 30. The tubing 31 is sized to make an outer diameter greater than the diameter of the hole 24 through the detachable dilator 20 and may be used to gently nudge the detachable dilator 20 off of the catheter 50, for example. Then, the needle 30 may be extracted from the catheter 50, leaving the dilator 20 to be retrieved by securing the loop 17 to a tool 151, such as illustrated in FIG. 9, for example.

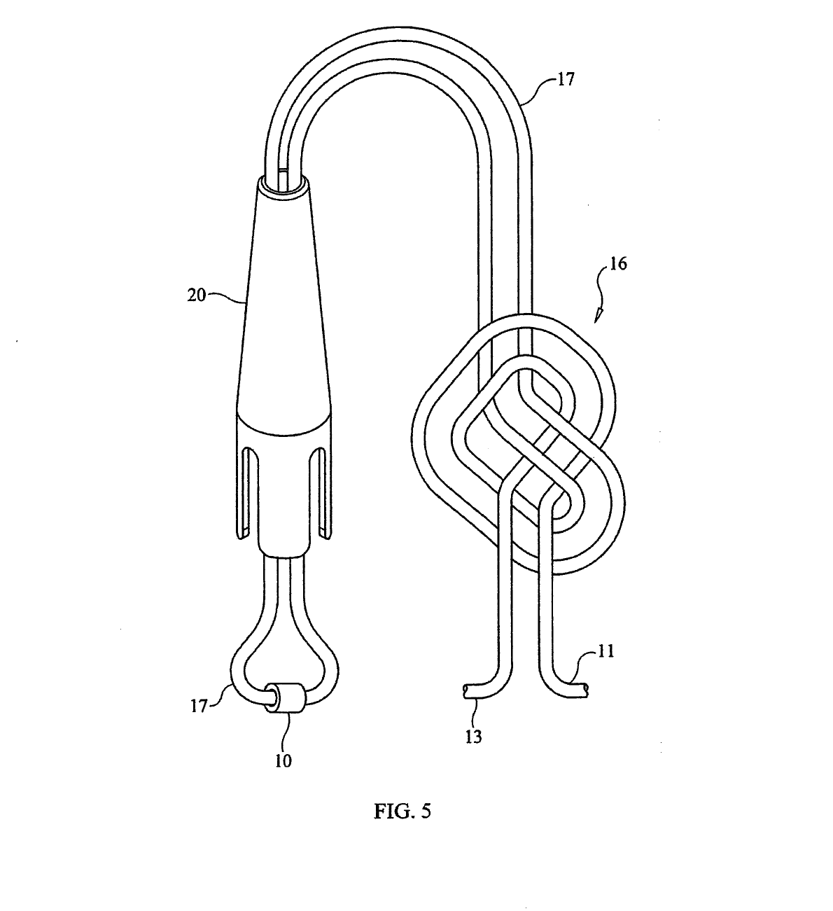

FIG. 5 illustrates yet another example of a bead 10 engaged by a string 12. One of the free ends 11, 13 are passed through the hole in the bead 10 before both ends are passed through the hole in the detachable dilator 20. Then, the free ends 11, 13 are tied in a knot 16 forming the loop 17.

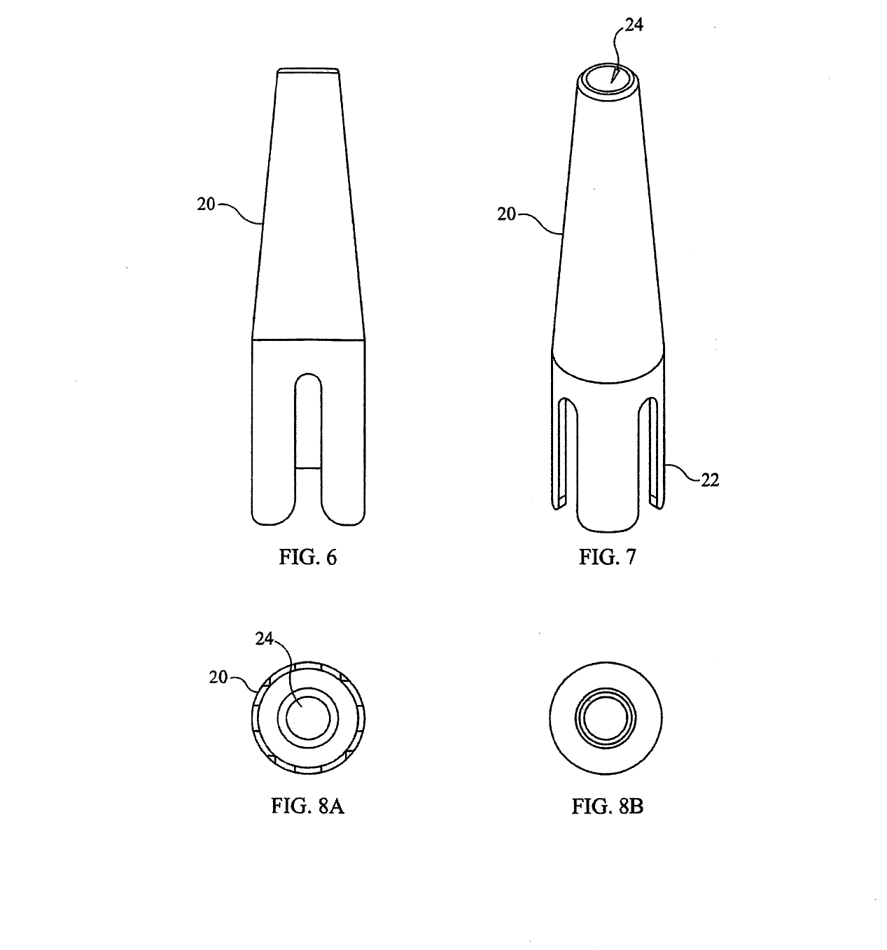

FIGS. 6-8B illustrate various views of the dilator 20, which may have a hole 24 through the dilator 20 and may have a collar 22, which may be formed a plurality of segmented collar portions 22. The dilator may be conical or any other shape. A conical shape helps during insertion to dilate the tissue through which the dilator 20, and the catheter to which the dilator is coupled, must pass. As illustrated in FIG. 3, segmented portions of the collar 22 may extend over a portion of the catheter 50, such as for protecting an expandable balloon or a portion of an expandable balloon during insertion.

In the example in FIG. 9, a safety hook 151 includes a safety catch 153 coupled to the hook 152 by a spring hinge 154. The hook 152 is arranged and configured to easily capture the loop 17 extending from a detachable dilator 20. The safety catch 153 easily pivots at the spring hinge 154, allowing the loop 17 to enter the hook 152, but the safety catch 153 prevents the loop 17 from disengaging from the hook 152, because a bias force keeps the safety catch 153 in position, as illustrated in FIG. 9. The safety hook 151 is coupled to a cable 156 by a threadingly engageable connector 155, for example. The tool 151 may be passed through an endoscope 150, for example, and may be retracted back into the endoscope, bringing the detachable dilator 20 into close proximity with the end of the endoscope 150 prior to withdrawing the endoscope 150 from the patients body. In this way, the dilator 20 may be retrieved from the patient's body, for example.

This detailed description provides examples including features and elements of the claims for the purpose of enabling a person having ordinary skill in the art to make and use the inventions recited in the claims. However, these examples are not intended to limit the scope of the claims, directly. Instead, the examples provide features and elements of the claims that, having been disclosed in these descriptions, claims and drawings, may be altered and combined in ways that are known in the art.

What is claimed is:

1. A catheter for percutaneous endoscopic catheterization for use with an endoscopically introduced device, the catheter comprising:
a catheter tube, having a central channel extending through the tube from a proximal end of the tube to a distal end of the tube;
a detachable dilator having a central channel extending through the dilator from a distal end of the dilator to a proximal end of the dilator, opposite of the distal end of the dilator, the proximal end of the dilator fitting onto the distal end of the tube such that the central channel of the dilator aligns with the central channel of the tube; and
an extraction device comprising a string, the string having a proximal portion and a distal portion, and a bead, the bead being fixed on the proximal portion of the string, wherein the proximal portion of the string and the bead are arranged within the catheter at the proximal end of the dilator, opposite of the distal end of the dilator, and at least a portion of the distal portion of the string extends from the distal end of the dilator, and the bead retains the proximal portion of the string within the dilator when the at least a portion of the distal portion of the string is secured by the endoscopically introduced device for extraction of dilator.

2. The catheter of claim 1, wherein the bead has a body and a hole extending through the body through which the proximal portion of the string extends, and the body of the bead is not formed by knotting the string.

3. The catheter of claim 2, wherein the body of the bead is cylindrical and the hole extending through the body is disposed along a longitudinal axis of the cylindrical body.

4. The catheter of claim 2, wherein the string is strung through the hole in the body of the bead.

5. The catheter of claim 2, wherein an outer dimension of the bead is greater than a dimension of the central channel extending through the dilator.

6. The catheter of claim 1, wherein the bead is made of a silicone.

7. The catheter of claim 1, wherein the string is an elongated wire, a suture, a line, a cord, a thread, a strip, or a fiber.

8. The catheter of claim 7, wherein the string is a suture material.

9. The catheter of claim 8, wherein the string has an average diameter from 0.044 mm to 0.45 mm.

10. The catheter of claim 8, wherein the suture material is knotted such that a knot fixes the string on the bead.

11. The catheter of claim 10, wherein the knot-pull strength of the string is from 0.09 kg to 5.23 kg.

12. The catheter of claim 11, wherein the distal end of the string forms a loop extending from the dilator.

13. The catheter of claim 1, wherein the bead is crimped or adhered onto the string.

14. The catheter of claim 13, wherein the bead is crimped onto the string.

15. The catheter of claim 13, wherein the string is not knotted.

16. The catheter of claim 9, wherein the suture material is of a monofilament polypropylene, a braided polyester or a braided silk.

17. The catheter of claim 1, wherein the bead is formed by knotting the string.

18. The catheter of claim 17, wherein the bead is a double overhand stopper knot.

19. A catheter kit comprising:
an endoscopically introduced device, wherein the endoscopically introduced device comprises a safety hook, having a hook portion and a safety catch portion; and
a catheter for percutaneous endoscopic catheterization, the catheter comprising:
a catheter tube, having a central channel extending through the tube from a proximal end of the tube to a distal end of the tube;
a detachable dilator having a central channel extending through the dilator from a distal end of the dilator to a proximal end of the dilator, opposite of the distal end of the dilator, the proximal end of the dilator fitting onto the distal end of the tube such that the central channel of the dilator aligns with the central channel of the tube; and
an extraction device comprising a string, the string having a proximal portion and a distal portion, and a bead, the bead being fixed on the proximal portion of the string, wherein the proximal portion of the string and the bead are arranged within the catheter at the proximal end of the dilator, opposite of the distal end of the dilator, and at least a portion of the distal portion of the string extends from the distal end of the dilator, and the bead retains the proximal portion of the string within the dilator when the at least a portion of the distal portion of the string is secured by the endoscopically introduced device for extraction of dilator, wherein the distal portion of the string forms a loop and the safety hook is arranged such that the loop passes between the hook portion and the safety catch portion of the safety hook, when the safety hook snares the loop, but the arrangement of the safety catch portion and hook portion of the safety hook prevents the loop from disengaging from the safety hook during extraction of the dilator from a patient.

20. A method for extraction of the dilator of the catheter of claim 1 from a patient, the method comprising:
endoscopically introducing the endoscopically introduced device, such that the endoscopically introduced device is disposed adjacent to the dilator;
securing the distal portion of the string extending from the dilator to the device; and
withdrawing the endoscopically introduced device, the dilator and the extraction device from the patient.
